(19) United States
(12) Patent Application Publication
(10) Pub. No.: US 2026/0091389 A1
(43) Pub. Date: Apr. 2, 2026

(54) MICROFLUIDIC ON-CHIP FILTERS

(71) Applicant: 10x Genomics, Inc., Pleasanton, CA (US)

(72) Inventors: Jorge BERNATE, Pleasanton, CA (US); Rajiv BHARADWAJ, Pleasanton, CA (US); Donald A. MASQUELIER, Pleasanton, CA (US); Tobias Daniel WHEELER, Pleasanton, CA (US)

(21) Appl. No.: 19/319,160

(22) Filed: Sep. 4, 2025

Related U.S. Application Data
(63) Continuation of application No. 16/180,378, filed on Nov. 5, 2018, now Pat. No. 12,427,518.
Application No. 16/180,378 is a continuation of application No. PCT/US2017/032525, filed on May 12, 2017.
(60) Provisional application No. 62/335,216, filed on May 12, 2016.

(51) Int. Cl. B01L 3/00; C12M 1/12; C12M 3/06; C12N 1/02; C12N 11/04

(52) U.S. Cl. CPC B01L 3/502753; B01L 3/502761; C12M 23/16; C12M 25/04; C12N 1/02; C12N 11/04; B01L 2200/0631; B01L 2200/0647; B01L 2300/021; B01L 2300/0681; B01L 2300/0816

(57) ABSTRACT

A microfluidic system for manipulating particles in a fluid is provided. The system includes a microfluidic chip having at least one channel and at least one filter feature, wherein the filter feature includes a field of physical obstacles configured and arranged to remove physical contaminants associated with the particles. The filter features are particularly suited to remove contaminants associated with gel beads manipulated by microfluidic handling.

### CROSS-REFERENCE

This Application is a Continuation of application Ser. No. 16/180,378 filed on Nov. 5, 2018. Application Ser. No. 16/180,378 is a Continuation of Application PCT/US2017/032525 filed on May 12, 2017. Application PCT/US2017/032525 claims the benefit of U.S. Provisional Application 62/335,216 filed on May 12, 2016. The entire contents of these applications are incorporated herein by reference in their entirety.

### BACKGROUND

Microfluidic systems may be used in various applications, such as biological sample processing, for example, for generating monodisperse populations of microcapsules or beads that may have reagents such as biological reagents associated therewith. Further, the microfluidic systems may be used for selectively and controllably partitioning these microcapsules or beads into droplets in emulsions for use in performing further reactions and/or analyses. Such systems may include a network of channels, and actuators for directing fluids through the network of channels. The network of channels can further comprise one or more flow controlling structures that provide a flow rate.

A microfluidic system may include a repository of biological material in fluid communication with a first channel and a micropump for subjecting the biological material to flow through the channel. The biological material, while flowing through the first channel, may be subjected to reagents in a second channel for generating emulsion at an intersection of the first channel and the second channel. The microfluidic system may be fluidly connected to an output reservoir for collecting a released biological material that was subjected to flow through the channel. The released biological material may comprise droplets in emulsions.

A microfluidic system may be integrated in a chip. The chip may be used to process a biological sample. The chip may further comprise a plurality of compartments. At least one compartment may be in fluid communication with the repository of biological material that may or may not be associated with the reagents. At least one compartment may be in fluid communication with the output reservoir. The plurality of compartments may be in communication with each other via the network of channels. The chip may be compatible with a plurality of devices for further analyzing the processed biological material. The plurality of devices may comprise high throughput next generation sequencing platforms, nucleic acid amplification systems and nucleic acid manipulation systems.

### SUMMARY

Such systems, including microfluidic channels and features, have proven useful tools for fluid and particle manipulations in a variety of applications. As microfluidic channel dimensions decrease, however, the likelihood of blockage in such channels (i.e., blocking rate or clogging rate) may increase. Such blockage can occur due to debris from consumables (e.g., pipette tips and reagent troughs), environment (e.g., airborne particulate that settles into chip wells) or due to debris/clumps in reagents themselves.

Such blockage can be an issue with gel bead emulsion reagent processing, for example, because (a) the gel bead solution may be a two-phase suspension flow that is more susceptible to blockage, and (b) the gel bead solution may have more debris compared to other single phase reagents (e.g., oil, aqueous solutions) due to the large number of processing steps involved in generating gel beads. Accordingly, recognized herein is the need for on-chip solutions to reduce blocking of microfluidic systems employing particles and emulsions, for example, gel bead emulsions.

In an aspect, the present disclosure provides a microfluidic device for filtering particles in a fluid. The microfluidic device comprises (i) a reservoir for holding the fluid comprising the particles; (ii) a channel in fluid communication with the reservoir; and (iii) at least one filter feature in the channel, where the at least one filter feature comprises a field of a plurality of physical obstacles configured and arranged in the channel, where the at least one filter feature separates the particles in the fluid upon flow of the fluid from the reservoir through the channel, and where the channel comprises a first end and a second end, where the first end is adjacent to the reservoir, and where a cross-section of the channel decreases along a direction leading from the first end to the second end.

In some embodiments, the reservoir, the channel and at least one filter feature are integrated in a chip. In some embodiments, the chip comprises a plurality of compartments in fluid communication with the reservoir. In some embodiments, at least one compartment of the plurality of compartments is in fluid communication with the reservoir, which reservoir includes the particles. In some embodiments, at least one compartment of the plurality of compartments is in fluid communication with an output reservoir that collects the particles.

In some embodiments, the plurality of physical obstacles is distributed in an array of physical obstacles. In some embodiments, the plurality of physical obstacles includes a plurality of cylinders each having a cross-sectional dimension and a vertical dimension arranged within the field. In some embodiments, cross-sectional dimensions and vertical dimensions of at least a subset of the plurality of cylinders are the same. In some embodiments, at least a subset of the plurality of cylinders have different cross-sectional dimensions and vertical dimensions.

In some embodiments, the plurality of cylinders arranged in a plurality of concentric rings where an innermost ring is in fluid communication with the reservoir. In some embodiments, the plurality of concentric rings comprises at least two rings. In some embodiments, the plurality of concentric rings comprises an inner ring and an outer ring. In some embodiments, the inner ring comprises a first subset of the plurality of cylinders having larger cross-sectional dimensions and/or vertical dimensions than a second subset of the plurality of cylinders in the outer ring.

In some embodiments, the plurality of cylinders are arranged in a serpentine configuration in fluid communication with the reservoir. In some embodiments, the serpentine configuration includes at least one convex portion comprising a subset of the plurality of cylinders, which convex portion is in fluid communication with the reservoir.

In some embodiments, the direction is oriented along a direction of a gravitational acceleration vector. In some embodiments, the direction is oriented along a direction of a pressure increase or decrease along a direction leading from the first end to the second end.

In some embodiments, the at least one filter feature comprises at least one passage with a cross-section that is greater than or equal to an average diameter of the particle in the fluid among other particles in the fluid. In some embodiments, the at least one filter feature comprises at least one passage with a cross-section that is less than an average diameter of the particle in the fluid among other particles in the fluid.

In some embodiments, the particles are contaminants. In some embodiments, the particles are beads. In some embodiments, the particles are gel beads. In some embodiments, the particles are cellular components. In some embodiments, the particles are cells. In some embodiments, the cells include or are enclosed in a gel or a polymer matrix. In some embodiments, the cellular components include one or more members selected from the group consisting of cell membrane, cell wall, organelles, macromolecular constituent(s), deoxyribonucleic acid (DNA), and ribonucleic acid (RNA).

In some embodiments, the particles are comprised in a population of particles. In some embodiments, the population of particles is homogeneous. In some embodiments, the population of particles is heterogeneous.

In some embodiments, the particles include barcodes coupled thereto. In some embodiments, the barcodes are nucleic acid molecules comprising nucleic acid sequences. In some embodiments, the barcodes across at least a subset of the particles are different.

In some embodiments, the channel comprises at least one constriction between the first end and the second end to reduce a concentration of air trapped in the filter feature. In some embodiments, the channel comprises at least two constrictions on opposite sides of the channel between the first end and the second end to reduce a concentration of air trapped in the filter feature. In some embodiments, the channel comprises at least one passive valve and at least one rectifier and the at least one filter feature is between the at least one passive valve and the at least one rectifier.

In an additional aspect, the present disclosure provides a system for filtering particles in a fluid. The system comprises a microfluidic device comprising (i) a reservoir for holding the fluid comprising the particles, (ii) a channel in fluid communication with the reservoir, and (iii) at least one filter feature in the channel, where the at least one filter feature comprises a field of a plurality of physical obstacles configured and arranged in the channel, where the at least one filter feature separates the particles in the fluid upon flow of the fluid from the reservoir through the channel, and where the channel comprises a first end and a second end, where the first end is adjacent to the reservoir, and where a cross-section of the channel decreases along a direction leading from the first end to the second end; and a controller operatively coupled to the microfluidic device, where the controller subjects the fluid to flow from the reservoir to the channel.

In some embodiments, the reservoir, the channel and at least one filter feature are integrated in a chip. In some embodiments, the chip comprises a plurality of compartments in fluid communication with the reservoir. In some embodiments, at least one compartment of the plurality of compartments is in fluid communication with the reservoir, which reservoir includes the particles. In some embodiments, at least one compartment of the plurality of compartments is in fluid communication with an output reservoir that collects the particles.

In some embodiments, the plurality of physical obstacles is distributed in an array of physical obstacles. In some embodiments, the plurality of physical obstacles includes a plurality of cylinders each having a cross-sectional dimension and a vertical dimension arranged within the field. In some embodiments, cross-sectional dimensions and vertical dimensions of at least a subset of the plurality of cylinders are the same. In some embodiments, at least a subset of the plurality of cylinders have different cross-sectional dimensions and vertical dimensions.

In some embodiments, the plurality of cylinders arranged in a plurality of concentric rings where an innermost ring is in fluid communication with the reservoir. In some embodiments, the plurality of concentric rings comprises at least two rings. In some embodiments, the plurality of concentric rings comprises an inner ring and an outer ring. In some embodiments, the inner ring comprises a first subset of the plurality of cylinders having larger cross-sectional dimensions and/or vertical dimensions than a second subset of the plurality of cylinders in the outer ring.

In some embodiments, the plurality of cylinders are arranged in a serpentine configuration in fluid communication with the reservoir. In some embodiments, the serpentine configuration includes at least one convex portion comprising a subset of the plurality of cylinders, which convex portion is in fluid communication with the reservoir.

In some embodiments, the direction is oriented along a direction of a gravitational acceleration vector. In some embodiments, the direction is oriented along a direction of a pressure increase or decrease along a direction leading from the first end to the second end.

In some embodiments, the at least one filter feature comprises at least one passage with a cross-section that is greater than or equal to an average diameter of the particle in the fluid among other particles in the fluid. In some embodiments, the at least one filter feature comprises at least one passage with a cross-section that is less than an average diameter of the particle in the fluid among other particles in the fluid.

In some embodiments, the particles are contaminants. In some embodiments, the particles are beads. In some embodiments, the particles are gel beads. In some embodiments, the particles are cellular components. In some embodiments, the particles are cells. In some embodiments, the cells include or are enclosed in a gel or polymer matrix. In some embodiments, the cellular components include one or more members selected from the group consisting of cell membrane, cell wall, organelles, macromolecular constituent(s), deoxyribonucleic acid (DNA), and ribonucleic acid (RNA).

In some embodiments, the particles are comprised in a population of particles. In some embodiments, the population of particles is homogeneous. In some embodiments, the population of particles is heterogeneous.

In some embodiments, the particles include barcodes coupled thereto. In some embodiments, the barcodes are nucleic acid molecules comprising nucleic acid sequences. In some embodiments, the barcodes across at least a subset of the particles are different.

In some embodiments, the channel comprises at least one constriction between the first end and the second end to reduce a concentration of air trapped in the filter feature. In some embodiments, the channel comprises at least two constrictions on opposite sides of the channel between the first end and the second end to reduce a concentration of air trapped in the filter feature. In some embodiments, the channel comprises at least one passive valve and at least one rectifier and the at least one filter feature is between the at least one passive valve and the at least one rectifier.

In an additional aspect, the present disclosure provides a method for filtering particles in a fluid. The method comprises (a) activating a microfluidic device comprising (i) a reservoir for holding the fluid comprising the particles, (ii) a channel in fluid communication with the reservoir, and (iii) at least one filter feature in the channel, where the at least one filter feature comprises a field of a plurality of physical obstacles configured and arranged in the channel, where the channel comprises a first end and a second end, where the first end is adjacent to the reservoir, and where a cross-section of the channel decreases along a direction leading from the first end to the second end; and (b) subjecting the fluid to flow from the reservoir to the channel, where upon flow of the fluid from the reservoir through the channel, the at least one filter feature separates the particles in the fluid.

In some embodiments, the method further comprises collecting the particles in an output reservoir. In some embodiments, the method further comprises directing the particles collected in the output reservoir to further processing. In some embodiments, the method further comprises directing the particles to partitions. In some embodiments, the partitions are droplets. In some embodiments, the partitions are wells.

In some embodiments, the particles include contaminants. In some embodiments, the particles include beads. In some embodiments, the particles include cellular components. In some embodiments, the particles include cells. In some embodiments, the cellular components include one or more members selected from the group consisting of cell membrane, cell wall, organelles, macromolecular constituent(s), deoxyribonucleic acid (DNA), and ribonucleic acid (RNA).

In some embodiments, the particles include barcodes coupled thereto. In some embodiments, the barcodes are nucleic acid molecules comprising nucleic acid sequences. In some embodiments, the barcodes across at least a subset of the particles are different.

In some embodiments, the fluid is subjected to flow with the aid of a pressure increase or decrease along a direction leading from the first end to the second end.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 schematically illustrates an example microfluidic device with a plurality of compartments.

FIG. 2 schematically illustrates an example microfluidic device with a plurality of compartments.

FIGS. 3A-3C provides photomicrographs of an example microfluidic device.

FIGS. 4A-4B provides photomicrographs of an example channel blocked by a fiber.

FIGS. 5A-5C schematically illustrates an example of at least one filter feature integrated with at least one passive valve and at least one rectifier in a channel.

FIG. 6 provides a photomicrograph showing top view of an example of at least one filter feature in a channel with constrictions on opposing sides of the channel.

FIG. 7 provides a photomicrograph showing top view of an example of at least one filter feature in a channel with constrictions on opposing sides of the channel and the at least one filter feature in fluid communication with a reservoir.

FIG. 8 schematically illustrates an example of at least one filter feature in a concentric rings arrangement in a first end in a channel, in fluid communication with a reservoir.

FIGS. 9A-9C schematically illustrates an example of at least one filter feature in a concentric rings arrangement with two configurations and provides dimensions for parts of the concentric rings arrangements.

FIG. 10 provides a photomicrograph of an example channel with at least one filter feature in a concentric rings arrangement in fluid communication with a reservoir holding a fluid with gel beads.

FIG. 11 schematically illustrates an example of at least one filter feature in a serpentine arrangement in a first end in a channel, in fluid communication with a reservoir.

FIGS. 12A-12B schematically illustrates an example of at least one filter feature in a serpentine arrangement with two configurations and provides dimensions for parts of the serpentine arrangements.

FIG. 13 provides a photomicrograph of an example of at least one filter feature in a serpentine arrangement in fluid communication with a reservoir holding a fluid with gel beads.

FIG. 14 shows an example computer control system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The term “barcode,” as used herein, generally refers to a label, or identifier, that conveys or is capable of conveying information about an analyte. A barcode can be part of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include: polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads in real time.

The term “genome,” as used herein, generally refers to genomic information from a subject, which may be, for example, at least a portion or an entirety of a subject's hereditary information. A genome can be encoded either in DNA or in RNA. A genome can comprise coding regions that code for proteins as well as non-coding regions. A genome can include the sequence of all chromosomes together in an organism. For example, the human genome has a total of 46 chromosomes. The sequence of all of these together may constitute a human genome.

The terms “adaptor(s)”, “adapter(s)” and “tag(s)” may be used synonymously. An adaptor or tag can be coupled to a polynucleotide sequence to be “tagged” by any approach including ligation, hybridization, or other approaches.

The term “sequencing,” as used herein, generally refers to methods and technologies for determining the sequence of nucleotide bases in one or more polynucleotides. The polynucleotides can be, for example, deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina, Pacific Biosciences, Oxford Nanopore, or Life Technologies (Ion Torrent). As an alternative, sequencing may be performed using nucleic acid amplification, polymerase chain reaction (PCR) (e.g., digital PCR, quantitative PCR, or real time PCR) or isothermal amplification. Such devices may provide a plurality of raw genetic data corresponding to the genetic information of a subject (e.g., human), as generated by the device from a sample provided by the subject. In some situations, systems and methods provided herein may be used with proteomic information.

The term “bead,” as used herein, generally refers to a particle. The bead may be a solid or semi-solid particle. The bead may be a gel bead. The bead may be formed of a polymeric material. The bead may be magnetic or non-magnetic.

The term “sample,” as used herein, generally refers to a biological sample of a subject. The biological sample may be a nucleic acid sample or protein sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swap. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

The term “biological particle,” as used herein, generally refers to a discrete biological system derived from a biological sample. The biological particle may be a virus. The biological particle may be a cell or derivative of a cell. The biological particle may be an organelle. The biological particle may be a rare cell from a population of cells. The biological particle may be any type of cell, including without limitation prokaryotic cells, eukaryotic cells, bacterial, fungal, plant, mammalian, or other animal cell types, mycoplasmas, normal tissue cells, tumor cells, or any other cell type, whether derived from single cell or multicellular organisms. The biological particle may be obtained from a tissue of a subject. Biological particles may be disruptable particles.

The biological particle may be a hardened cell. Such hardened cell may or may not include a cell wall or cell membrane. The biological particle may include one or more constituents of a cell, but may not include other constituents of the cell. A cell may be a live cell. The live cell may be capable of being cultured, for example, being cultured when enclosed in a gel or polymer matrix, or cultured when comprising a gel or polymer matrix.

The term “macromolecular constituent,” as used herein, generally refers to a macromolecule contained within a biological particle. The macromolecular constituent may comprise a nucleic acid. The macromolecular constituent may comprise deoxyribonucleic acid (DNA). The macromolecular constituent may comprise ribonucleic acid (RNA). The macromolecular constituent may comprise a protein. The macromolecular constituent may comprise a peptide. The macromolecular constituent may comprise a polypeptide.

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise an oligonucleotide or polypeptide sequence. The molecular tag may comprise a DNA aptamer. The molecular tag may be or comprise a primer. The molecular tag may be or comprise a protein. The molecular tag may comprise a polypeptide. The molecular tag may be a barcode.

The efficiency of many single cell applications can increase by improving cell throughput. For example, this can be achieved by sorting a plurality of droplets that may or may not contain cells and/or particles therein to collect only the droplets that contain the cells and/or particles therein. The isolated population of droplets that contain the cells and/or particles therein can then be subject to further applications, such as nucleic acid amplification and/or sequencing applications.

### Apparatuses and Systems

In an aspect, the present disclosure provide a microfluidic device for filtering particles in a fluid. The microfluidic device may include a reservoir for holding the fluid comprising the particles, a channel in fluid communication with the reservoir, at least one filter feature in the channel. The at least one filter feature may comprise a field of a plurality of physical obstacles configured and arranged in the channel. The at least one filter feature may separate the particles in the fluid upon flow of the fluid from the reservoir through the channel.

For example, the at least one filter feature may retain the particles and permit the fluid to flow through, thereby separating the particles from the fluid. As another example, the at least one filter feature may separate a first subset of particles (e.g., gel beads) from a second subset of particles (e.g. contaminants or debris), to permit the fluid with the first subset of particles to flow through with a decrease in an amount or concentration of the second subset of particles or contaminants or debris.

The channel may comprise a first end and a second end, wherein the first end is adjacent to the reservoir. A cross-section of the channel may decrease along a direction leading from the first end to the second end. The cross-section may be tapered or chamfered. For example, the cross-section gradually decreases.

The reservoir, the channel and the at least one filter feature may be integrated in a chip. For example, the chip may include the reservoir, the channel and the at least one filter feature as a single piece unit. The chip may comprise a plurality of compartments in fluid communication with the reservoir. At least one compartment of the plurality of compartments may be in fluid communication with the reservoir. The reservoir may include the particles.

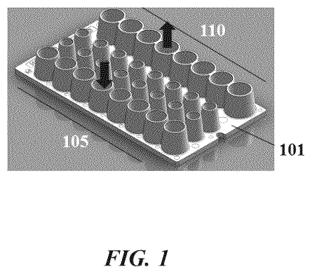

As shown in FIG. 1, a microfluidic device 101 may comprise a plurality of compartments. The plurality of compartments may further comprise a first subset of compartments 105 and a second subset of compartments 110. The first subset of compartments 105 may be in fluid communication with a reservoir (not shown). The reservoir may include the particles in a fluid. The first subset of compartments 105 may receive the fluid with the particles from the reservoir. The first subset of compartments 105 may direct the fluid through a network of channels (not shown) in the microfluidic device 101. The network of channels may connect to the first subset of compartments 105 with a first end and may further connect to the second subset of compartments 110 with a second end.

The second set of compartments 110 may be in fluid communication with an output reservoir (not shown). The fluid comprising the particles (e.g., cells) may be subjected to filtration in the channels. The channels may comprise the at least one filter feature (not shown). The at least one filter feature may filter the cells that may further be collected through the second set of compartments 110. The cells after filtration may be released in the output reservoir and the cells may then be collected from the output reservoir. The output reservoir may hold the particles for further processing, for example, sample preparation for assaying (e.g., sequencing). The output reservoir may direct the particles to partitions, such as to droplets and wells. The wells may further include an array of wells on a plate.

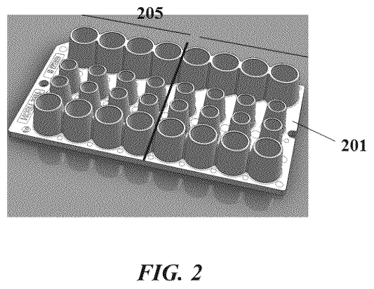

In another example, as shown in FIG. 2, the microfluidic device 201 may comprise a plurality of compartments. The plurality of compartments may further comprise a first subset of compartments 205 and a second subset of compartments 210. The first subset of compartments 205 may be in fluid communication with a reservoir (not shown) and may receive a fluid with particles (e.g., cells and a gel or a matrix) from the reservoir to produce cell beads. The cell beads may be directed to the second subset of compartments 210 for filtering the cell beads. A first end of a network of channels (not shown) may be in fluid communication with the first subset of compartments 205 and may receive the cell beads. The network of channels may carry the cell beads for filtration to the second subset of compartments 210. A second end of the network of channels may be in fluid communication with the second subset of compartments 210. The cell beads may be collected from the second set of compartments 210 after filtration. The cell beads may be released in an output reservoir (not shown) after filtration. The cells beads may be collected from the output reservoir. The output reservoir may hold the particles for further processing, for example, sample preparation for assaying (e.g., sequencing). The output reservoir may direct the particles to partitions, such as to droplets and wells. The wells may further include an array of wells on a plate.

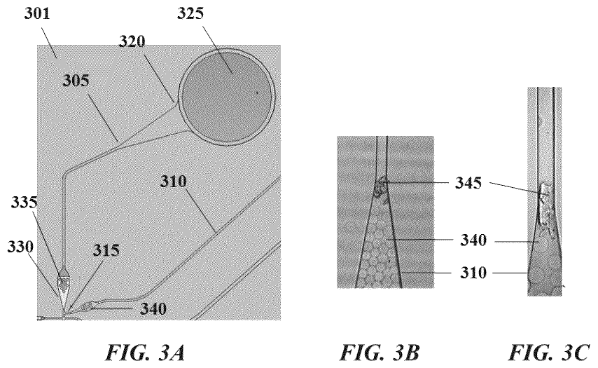

In another example, as shown in FIG. 3, the microfluidic device 301 may comprise a network of channels. The network of channels may comprise a first channel 305 and a second channel 310 that may intersect at an intersection 315. A first end 320 of the first channel may be in fluid communication with a reservoir 325, and a second end 330 of the first channel may be in fluid communication with the second channel 310 at the intersection 315. The first end 320 of the first channel 305 may taper toward the second end 330 of the first channel. For example, a cross-sectional dimension of the first end 320 may be larger than a cross-sectional dimension of the second end 330. The first channel 305 may carry a fluid with particles (e.g., debris or fibers or contaminants). The second end 330 of the first channel 305 may comprise at least one filter feature 335 that may separate the particles in the fluid before fluidly connecting with the second channel 310. The second channel 310 may comprise at least one filter feature (not shown). The at least one filter feature in the second channel 310 may reduce concentration of particles (e.g. debris) associated with the gel beads 340. The second channel 310 may not comprise the at least one filter feature and may be subjected to blockage by the particles (e.g. debris) 345 associated with the gel beads. The blockage in the second channel 310 may affect flow of the gel beads to the intersection 315.

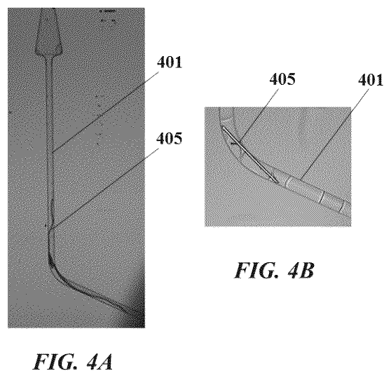

In another example, as shown in FIG. 4A-B, a channel 401 carrying a fluid may be blocked by particles (e.g., fibers) 405. Fibers 405 may block the channel 401 and may affect flow of the fluid.

The at least one filter feature may be located at the first end of the channel, also called as a “funnel filter”. The at least one filter feature may be located at the second end of the channel, also called as a “channel filter”. The funnel filter in the channel may be useful in separating particles that are larger in size and are rigid, such as, fibers. The channel filter may be useful in separating particles that are smaller in size, such as contaminants in reagents.

The at least one filter feature may comprise a plurality of physical obstacles. The plurality of obstacles may be located on both the first end and the second end. For example, the channel may comprise both the funnel filter and the channel filter. The plurality of obstacle may be located at either the first end or the second end of the channel. For example, the channel may comprise either the funnel filter or the channel filter. The channel may include a plurality funnel filters. The channel may include a plurality of channel filters. The may include combination of the plurality of funnel filters and the plurality of channel filters.

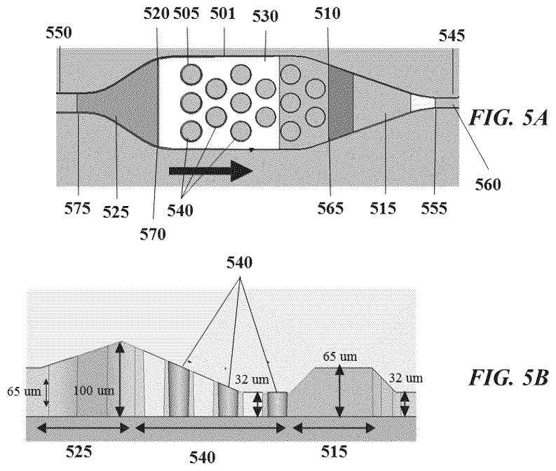

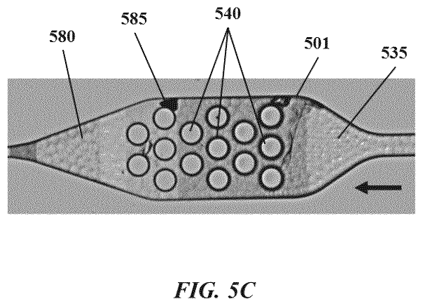

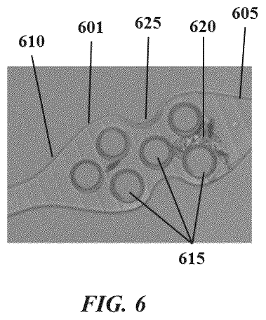

The at least one filter feature in a channel may be configured to reduce a concentration of air trapped in the at least one filter feature. For example, as shown in FIG. 5, the channel 501 may comprise at least one filter feature 505. A first end of at least one filter feature 510 may be in contact with at least one passive valve 515. A second end of at least one filter feature 520 may be in contact with at least one rectifier 525. The channel 501 may carry a fluid 530 with particles (e.g., gel beads, debris, fibers) 535 having a direction of flow as indicated by an arrow in FIG. 5A and FIG. 5C.

The at least one filter feature 505 may comprise a plurality of physical obstacles 540. The plurality of physical obstacles 540 may be arranged in a staggered arrangement. The plurality of physical obstacles 540 in the staggered arrangement may have alternating rows of odd and even number of the plurality of physical obstacles 540. The plurality of physical obstacles 540 may have a vertical dimension. The vertical dimension of the plurality of physical obstacles 540 may be identical. The vertical dimension of a given subset in the plurality of physical obstacles 540 may be different. For example, as shown in FIG. 5B, the vertical dimension of a first subset of the plurality of physical obstacles may be at least about 10 micrometer (μm), at least about 20 μm, at least about 30 μm, at least about 40 μm, at least about 50 μm, at least about 60 μm, at least about 100 μm or more. Further, the vertical dimension of a second subset of the plurality of physical obstacles may be at least about 60 μm. Furthermore, the vertical dimension of a second subset of the plurality of physical obstacles may be at least about 10 μm, at least about 25 pm, at least about 50 μm, at least about 60 μm, at least about 75 μm, at least about 100 μm, at least about 110 μm, at least about 125 um, at least about 150 μm, at least about 175 μm, at least about 200 μm or more.

The at least one passive valve 515 may regulate flow of the fluid 530 through the channel 501 in the microfluidic device. The at least one passive valve 515 may be configured to open when a fluid pressure in a first end of channel 545 is greater than fluid pressure in a second end of channel 550. In another example, the at least one passive valve 515 may be configured to open when a fluid pressure in a first channel is greater than a fluid pressure in a second channel (not shown in FIG. 5). A first end of at least one passive valve 555 may be in fluid communication with a reservoir 560. A second end of at least one passive valve 565 may be in fluid communication with the first end of at least one filter feature 510. The at least one passive valve 515 may regulate the fluid pressure entering the at least one filter feature 505. The at least one passive valve 515 may have a vertical dimension. The vertical dimension of the first end of at least one passive valve and the second end of at least one passive valve may be identical. The vertical dimension of the first end of at least one passive valve 555 and the second end of at least one passive valve 565 may be different. For example, as shown in FIG. 5B, the vertical dimension of the first end of at least one passive valve 555 may be narrower than the cross-sectional dimension of the second end of at least one passive valve 565. The vertical dimension of the first end of at least one passive valve 555 may be at least about 32 micrometer (μm) and the cross-sectional dimension of the second end of at least one passive valve 565 may be at least about 65 μm.

The second end of at least one filter feature 520 may be in contact with a first end of at least one rectifier 570. The at least one filter feature 505 may be in between the at least one passive valve 515 and the at least one rectifier 525. The at least one rectifier 525 may facilitate operation of the microfluidic device by allowing the fluid to traverse through the channel 501 from the first end of channel 545 toward the second end of channel 550. The at least one rectifier 525 may have different shapes, such as triangular, hyperbolic or other geometric shapes. The at least one rectifier 525 may have a vertical dimension. The vertical dimension of the first end of at least one rectifier and a second end of at least one rectifier may be identical. The vertical dimension of the first end of at least one rectifier 570 and the second end of at least one rectifier 575 may be different. For example, as shown in FIG. 5B, the vertical dimension of the first end of at least one rectifier 570 may be broader than the cross-sectional dimension of the second end of at least one rectifier 575. The vertical dimension of the first end of at least one rectifier 570 may be at least about 100 micrometer (μm) and the vertical dimension of the second end of at least one rectifier 575 may be at least about 65 μm.

The fluid with particles may flow through the plurality of physical obstacles in the directed indicated by the arrow in FIG. 5A and FIG. 5C. The particles may comprise a first subset of particles (e.g., gel beads) 580 and a second subset of particles (e.g., debris, fibers or contaminants) 585. The first subset of particles 580 may be released toward the second end of channel. The second subset of particles 585 may be trapped in the plurality of physical obstacles 540. The plurality of physical obstacles 540 may be useful in separating the second subset of particles 580 associated with the first subset of particles 585.

The channel comprising at least one filter feature may be configured to reduce a concentration of air trapped in the at least one filter feature. For example, as shown in FIG. 6, the channel 601 may include the at least one filter feature between a first end of channel 605 and a second end of channel 610. The at least one filter feature may comprise a plurality of physical obstacles 615. The plurality of physical obstacles 615 may be in a staggered arrangement. The first end of channel 605 may receive a fluid with particles 620 (e.g. debris or fibers). The second end of channel 610 may carry the fluid after filtering through the plurality of physical obstacles 615. The channel 601 may comprise at a plurality of constrictions 625 between the first end of channel 605 and a second end of channel 610. The plurality of constrictions 625 may be at least about 2, 3, 4, 5 or more. The plurality of constrictions may be at least about 2 constrictions on opposing sides of the channel 601. The fluid with particles 620 may flow through the plurality of physical obstacles from the first end of channel 605 toward the second end of channel 610. The particles 620 may be trapped in the plurality of physical obstacles 615. The plurality of physical obstacles 615 may be useful in separating the particles 620 in the fluid.

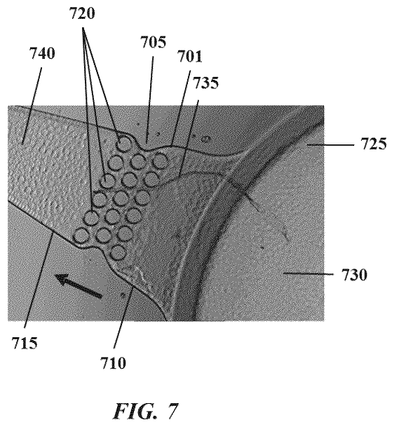

A channel with a plurality of constrictions may be used to separate a first subset of particles (e.g., gel beads) from a second subset of particles (e.g., debris, fibers). For example, as shown in FIG. 7, a channel 701 may comprise a plurality of constrictions 705 between a first end of channel 710 and a second end of channel 715. The channel 701 may also comprise a plurality of physical obstacles 720 between the first end of channel 710 and the second end of channel 715. The first end of channel 710 may be in fluid communication with a reservoir 725 holding a fluid with particles 730. The first end of channel 710 may receive the fluid. The fluid may then be filtered through the plurality of physical obstacles 720. The fluid may flow through the plurality of physical obstacles 720 as indicated by an arrow in FIG. 7. The plurality of physical obstacles 720 in the channel 701 may be in a staggered arrangement. The plurality of physical obstacles 720 may trap the second subset of particles 735 and the first subset of particles 740 may flow through. The plurality of constrictions 705 may reduce the concentration of air trapped in the plurality of physical obstacles 720.

The plurality of physical obstacles may be arranged in an array in a field in a channel. The plurality of physical obstacles may be distributed randomly in the field in the channel. The array of the plurality of physical obstacles may be in a staggered arrangement. The plurality of physical obstacles in the staggered arrangement may have alternating rows of odd and even number of the plurality of physical obstacles. The plurality of physical obstacles in the array may have a parallel arrangement. The plurality of physical obstacles in the parallel arrangement may form rows and columns with either odd or even number of plurality of physical obstacles.

The plurality of physical obstacles in the field may be at least about 1, 2, 3, 4, 5, 6, 7, 8, 9, 10, 15, 20, 30, 40, 50, 60, 70, 80, 90, 100 or more. In one example, the plurality of physical obstacles may be at least about 6. In another example, the plurality of physical obstacles may be at least about 15. In another example, the plurality of physical obstacles may be at least about 17.

The plurality of physical obstacles may have various shapes, such as cylindrical, conical or pyramidal. In some examples, the plurality of physical obstacles may be cylindrical. The plurality of physical obstacles may be coated with solution. In some examples, the plurality of physical obstacles may be coated with hydrophobic solution.

The plurality of physical obstacles may further comprise a plurality of cylinders. An individual cylinder in the plurality of cylinders may have a cross-sectional dimension and a vertical dimension. In some examples, cross-sectional dimensions and vertical dimensions of at least a subset of the plurality of cylinders may be identical. In other examples, cross-sectional dimensions and vertical dimensions of at least a subset of the plurality of cylinders may be the different.

The plurality of cylinders may comprise at least one passage between the plurality of cylinders. The at least one passage may have a cross-sectional dimension and a vertical dimension. In some examples, cross-sectional dimensions and vertical dimensions of at least a subset of the plurality of cylinders may be the identical. In other examples, cross-sectional dimensions and vertical dimensions of at least a subset of the plurality of cylinders may be the different.

The plurality of cylinders may be arranged in a plurality of concentric rings, sometimes referred to as concentric rings arrangement. The plurality of concentric rings may have an innermost ring. The innermost ring may be in fluid communication with the reservoir. The concentric rings arrangement may include at least about 2, 3, 4, 5, 6, 7, 8, 9, 10 or more of the plurality of concentric rings. The concentric rings arrangement may include at least about 2 of the plurality of concentric rings.

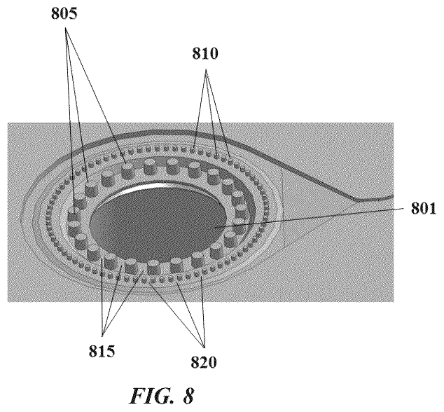

In some examples, as shown in FIG. 8, a funnel filter may be placed at a first end of a channel. The funnel filter may be in fluid communication with a reservoir 801. The funnel filter may comprise a plurality of physical obstacles. The physical obstacles may comprise a plurality of cylinders. The plurality of cylinders may be arranged in a plurality of concentric rings, sometimes referred to as a concentric rings arrangement. The plurality of concentric rings may be at least about 2. The plurality of concentric rings may comprise an inner ring and an outer ring.

The inner ring may include a first subset of plurality of cylinders 805 and the outer ring may include a second subset of plurality of cylinders 810. The first subset of plurality of cylinders 805 may be in fluid communication with a reservoir 801. The first subset of plurality of cylinders 805 may have larger cross-sectional dimensions and/or vertical dimensions than the second subset of plurality of cylinders 810. In some examples, the inner ring and the outer ring may include the plurality of cylinders having identical cross-sectional dimensions and/or vertical dimensions.

The plurality of cylinders may comprise at least one passage between the plurality of cylinders. The at least one passage may have a cross-sectional dimension and/vertical dimension. The first subset of plurality of cylinders 805 may comprise a first subset of passages 815. The second subset of plurality of cylinders 810 may comprise a second subset of passages 820. The first subset of passages 815 may have different cross-sectional dimensions and/vertical dimensions than the second subset of passages 820. The first subset of passages 815 and the second subset of passages may have identical cross-sectional dimensions and/vertical dimensions.

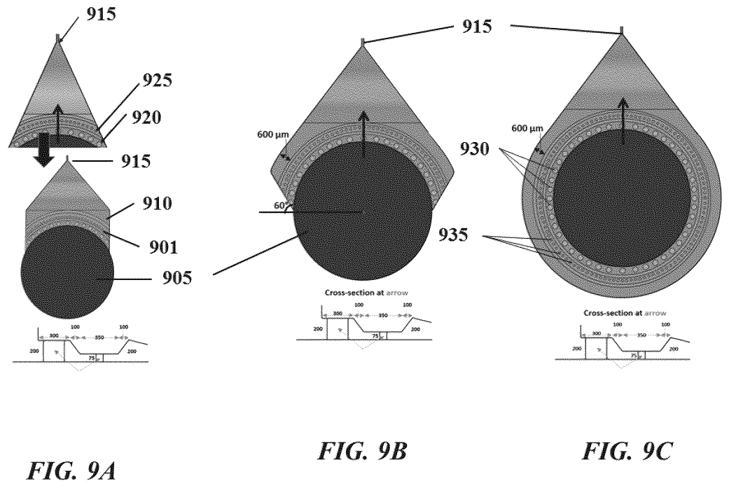

The cross-sectional dimension of the plurality of cylinders in the inner ring may be at least about 1 micrometer (μm), 10 μm, 20 μm, 30 μm, 40 μm, 50 μm, 100 μm, 150 μm, 160 μm, 170 μm, 180 μm, 190 μm, 200 μm, 300 μm or more. In one example, as shown in FIG. 9A-C the cross-sectional dimensional is at least about 300 μm. The vertical dimensional of the plurality of cylinders in the inner ring may be at least about 1 micrometer (um), 10 μm, 20 μm, 30 μm, 40 μm, 50 μm, 100 μm, 150 μm, 160 μm, 170 μm, 180 μm, 190 μm, 200 μm, 300 μm or more. In one example, as shown in FIG. 9A-C the vertical dimensional is at least about 200 μm.

The cross-sectional dimension of the plurality of cylinders in the outer ring may be at least about 1 micrometer (um), 10 μm, 20 μm, 30 μm, 40 μm, 50 μm, 100 μm, 150 μm, 160 μm, 170 μm, 180 μm, 190 μm, 200 μm or more. In one example, the cross-sectional dimensional is at least about 200 μm. The vertical dimension of the plurality of cylinders in the outer ring may be at least about 1 μm, 10 μm, 20 μm, 30 μm, 40 μm, 50 μm 60 μm, 70 μm, 80 μm, 90 μm, 100 μm or more. In one example, as shown in FIG. 9A-C the vertical dimensional is at least about 75 μm. The cross-sectional dimension of the plurality of cylinders in the inner ring may be greater than the cross sectional dimension of the plurality of cylinders in the outer ring.

The cross-sectional dimension of the first subset of passages may be at least about 1 micrometer (μm), 10 μm, 20 μm, 30 μm, 40 μm, 50 μm, 100 μm, 150 μm, 160 μm, 170 μm, 180 μm, 190 μm, 200 μm, 210 μm, 225 μm, 250 μm, 300 μm, 350 μm, 400 μm or more. In one example, the cross-sectional dimension of the first subset of passages may be at least about 200 μm. The cross-sectional dimension of the second subset of passages may be at least about 1 micrometer (μm), 10 μm, 20 μm, 30 μm, 40 μm, 50 μm, 100 μm, 150 μm, 160 μm, 170 μm, 180 μm, 190 μm, 200 μm or more. In one example, the cross-sectional dimension of the second subset of passages may be at least about 75 μm.

In some examples, as shown in FIG. 9A-C, at least one filter feature 901 in a concentric rings arrangement may encircle a reservoir 905. In one example, as shown in FIG. 9A-B, the at least one filter feature 901 in the concentric rings arrangement may encircle reservoir 905 partially. In another example, as shown in FIG. 9C, the at least one filter feature 901 in the concentric rings arrangement may encircle reservoir 905 completely. The at least one filter feature may be at a first end of channel 910, in fluid communication with the reservoir 905. The first end of channel 910 tapers toward a second end of channel 915. In one example, a cross-sectional dimension of the first end of channel 910 may be at least about 200 μm. A cross-sectional dimension of the second end of channel 915 may be at least about 75 μm. The concentric rings arrangement may comprise an inner ring 920 and an outer ring 925. The inner ring 920 may comprise a first subset of plurality of cylinders 930. The outer ring 925 may comprise a second subset of plurality of cylinders 935. Cross-sectional dimensions and/vertical dimensions of the first subset of plurality of cylinders 930 in the inner ring 920 may be greater than the cross sectional dimensions and/or vertical dimensions of the second subset of plurality of cylinders 935 in the outer ring 925.

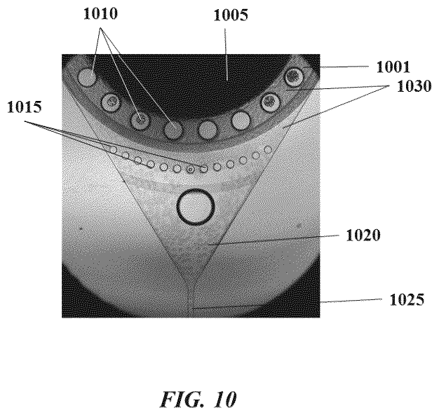

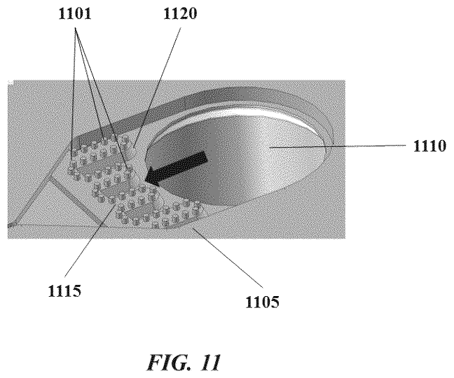

In some examples, as shown in FIG. 10, a concentric rings arrangement of at least one filter feature may be used to filter a fluid with particles (e.g., gel beads). The at least one filter feature may be located at a first end of channel 1001. The at least one filter feature may be configured to partially encircle a reservoir 1005 holding the fluid with particles. The concentric rings arrangement may comprise an inner ring and an outer ring. The inner ring may comprise a first subset of plurality of cylinders 1010. The outer ring may comprise a second subset of plurality of cylinders 1015. Cross-sectional dimensions and/vertical dimensions of the first subset of plurality of cylinders 1010 in the inner ring may be greater than the cross sectional dimensions and/or vertical dimensions of the second subset of plurality of cylinders 1015 in the outer ring.

The particles may comprise gel beads 1020. The gel beads 1020 in the fluid may flow from the first end of channel to a second end of channel 1025. The gel beads 1020 may form aggregates 1030 in the channel. The aggregates 1030 of the gel beads 1020 may be trapped in the first subset of plurality of cylinders 1010 and the second subset of plurality of cylinders 1015. The concentric rings arrangement may be useful in separating the aggregates 1030 from the gel beads 1020.

In some examples, as shown in FIG. 11, the plurality of cylinders 1101 may be arranged in a serpentine configuration. The plurality of cylinders 1101 may be located in a first end of channel 1105, in fluid communication with a reservoir 1110. The plurality of cylinders 1101 may partially encircle the reservoir 1110. The serpentine configuration may include at least one flat portion 1115 connecting to at least one convex portion 1120, protruding towards the reservoir 1110. The convex portion 1120 may have a drop-off with respect to the flat portion 1115. The at least one flat portion 1115 may connect the at least one convex portion 1120 on opposite ends of the at least one flat portion 1115. The at least one convex portion 1120 may come in contact with the fluid first as the fluid from the reservoir 1110 to the first end of channel 1105, as indicated by an arrow. The serpentine configuration may comprise at least about three flat portions connecting to at least about four convex portions. The arrow illustrates the general direction of fluid flow, in an example. In another example, the general direction of fluid flow may be reversed.

The serpentine configuration may comprise at least about 1, 2, 3, 4, 5, 6, 7, 8, 9, 10, 20, 30, 40, 50, 60, 70, 80, 90, 100 flat portions. The serpentine configuration may connect or be in fluid communication with at least 1, 2, 3, 4, 5, 6, 7, 8, 9, 20, 30, 40, 50, 60, 70, 80, 90, or 100 convex portions.

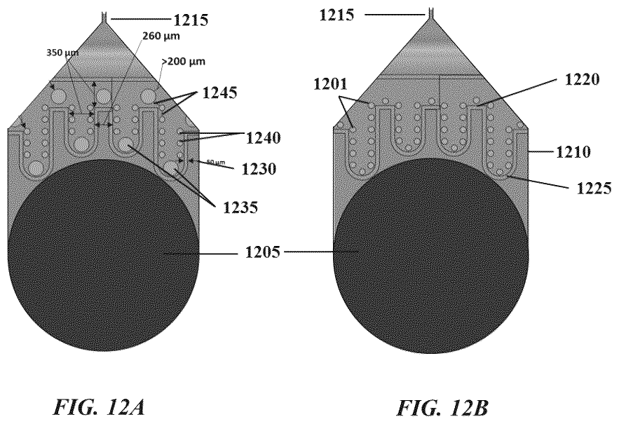

In one example, as shown in FIG. 12A, the plurality of cylinders 1201 in the serpentine configuration may partially encircle a reservoir 1205 at a first end of channel 1210. In other examples, the plurality of cylinders 1201 in the serpentine configuration may completely encircle the reservoir at the first end of channel (not shown).

The first end of channel 1210 tapers toward a second end of channel 1215. The first end of channel 1210 may have larger cross-sectional dimension than the second end of channel 1215. For example, the cross-sectional dimension of the first end of channel 1210 may be at least about 200 μm. The cross-sectional dimension of the second end of channel 1215 may be at least about 75 μm.

The serpentine configuration may include at least one flat portion 1220 connecting to at least one convex portion 1225, protruding towards the reservoir 1205. Cross-sectional dimensions of the at least one flat portion 1220 may be at least about 10 μm, 100 μm, 200 μm, 300 μm, 400 μm or more. In one example, as shown in FIG. 12A, a cross-sectional dimension of the at least one flat portion 1220 may be at least about 260 μm. Cross-sectional dimensions of the at least one convex portion 1225 may be at least about 10 μm, 100 μm, 200 μm, 300 μm, 400 μm or more. In one example, as shown in FIG. 12A, a cross-sectional dimension of the at least one convex portion 1225 may be at least about 350 μm. The at least one convex portion 1225 may have a wall 1230 having a cross-sectional dimension. The cross-sectional dimension of the wall 1230 may be at least about 1 μm, 10 μm, 20 μm, 30 μm, 40 μm or more. In one example, as shown in FIG. 12A, the cross-sectional dimension of the wall 1230 may be at least about 50 μm. The at least one convex portion 1225 may have vertical dimensions. As shown in FIG. 12A, the vertical dimensions of the at least one convex portion may be different. The vertical dimensions of the at least one convex portion may be similar (not shown).

The plurality of cylinders 1201 in the serpentine arrangement may have different cross-sectional dimensions and/or vertical dimensions. For example, as shown in FIG. 12A, the plurality of cylinders 1201 may have a first subset of cylinders 1235 and a second subset of cylinders 1240. The first subset of cylinders 1235 may be located at a tip of the at least one convex portion and at the at least one flat portion 1220. The first subset of cylinders 1235 may have larger cross-sectional dimensions than the second subset of cylinders 1240. Cross-sectional dimensions of the first subset of cylinders 1235 may be at least about 1 μm, 100 μm, 200 μm, 300 μm, 400 μm or more. In one example, as shown in FIG. 12A, a cross-sectional dimension of the first subset of cylinders 1235 may be at least about 200 μm. Cross-sectional dimensions of the second subset of cylinders 1240 may be at least about 1 μm, 100 μm, 200 μm, 300 μm, 400 μm or more. In one example, as in FIG. 12A, a cross-sectional dimension of the second subset of cylinders 1240 may be at least about 75 μm. Vertical dimensions of the first subset of cylinders 1235 may be at least about 1 μm, 100 μm, 200 μm, 300 μm, 400 μm or more. In one example, as in FIG. 12A, a vertical dimension of the first subset of cylinders 1235 may be at least about 200 μm. Vertical dimensions of the second subset of cylinders 1240 may be at least about 1 μm, 100 μm, 200 μm, 300 μm, 400 μm or more. In one example, as in FIG. 12A, a vertical dimension of the second subset of cylinders 1240 may be at least about 75 μm.

The plurality of cylinders 1201 in the serpentine arrangement may have identical cross-sectional dimensions and/or vertical dimensions. For example, as shown in FIG. 12B, cross-sectional dimensions of the plurality of cylinders 1201 may be at least about 1 μm, 100 μm, 200 μm, 300 μm, 400 μm or more. In one example, as shown in FIG. 12B, cross-sectional dimensions of the plurality of cylinders 1201 may be at least about 75 μm. Vertical dimensions of the plurality of cylinders 1201 may be at least about 1 μm, 100 μm, 200 μm, 300 μm, 400 μm or more. In one example, as in FIG. 12B, vertical dimensions of the plurality of cylinders 1201 may be at least about 75 μm.

The plurality of cylinders 1201 in the serpentine configuration may include at least one passage 1245. The at least one passage 1245 may have a cross-sectional dimension. The cross-sectional dimension of the at least one passage 1245may be similar. The cross-sectional dimension of the at least one passage 1245 may be at least about 1 μm, 5 μm, 10 μm, 20 μm, 30 μm, 40 μm, 50 μm, 60 μm, 70 μm, 80 μm, 90 μm, 100 μm, 110 μm, 120 μm, 130 μm, 140 μm, 150 μm or more. In one example, as shown in FIG. 12B, cross-sectional dimension of the at least one passage 1245 may be at least about 75 μm. The cross-sectional dimension of the at least one passage may be different (not shown).

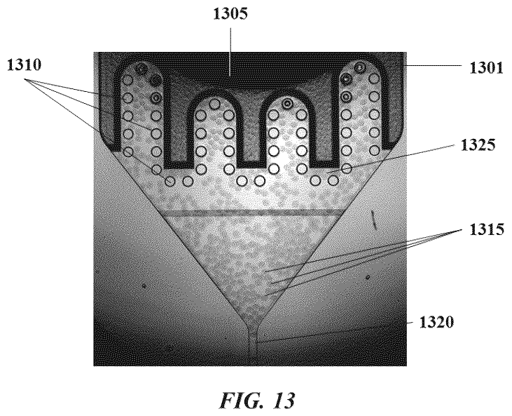

In some examples, as shown in FIG. 13, a serpentine arrangement of at least one filter feature may be used to filter a fluid with particles (e.g., gel beads). The at least one filter feature may be located at a first end of channel 1301. The at least one filter feature may be configured to partially encircle a reservoir 1305 holding the fluid with particles. The serpentine arrangement may comprise a plurality of cylinders 1310. Cross-sectional dimensions and/vertical dimensions of the plurality of cylinders 1310 in the serpentine ring arrangement may be similar. The particles may comprise gel beads 1315. The gel beads 1315 in the fluid may flow from the first end of channel to a second end of channel 1320. The gel beads 1315 may form aggregates 1325 in the channel. The aggregates 1325 of the gel beads 1315 may be trapped in the plurality of cylinders 1310. The serpentine arrangement may be useful in separating the aggregates 1325 from the gel beads 1315.

The direction of the fluid in the channel from the first end of channel to the second end of channel may be oriented along a direction of a gravitational acceleration vector. The direction of the gravitational acceleration vector may include positioning the first end above the second end such that the reservoir may be above the output reservoir.

The direction of the fluid in the channel from the first end to the second end may be oriented along a direction of a pressure increase or decrease along a direction leading from the first end to the second end. The direction of the pressure increase or decrease may include positioning of the first end and the second end such that first end may be higher pressure than the second end. The pressure increase or decrease may be achieved by using a controller coupled to a pump (e.g., micro pump).

The at least one filter feature may comprise at least one passage with a cross-section. The cross-section of the at least one passage may be greater than or equal to an average diameter of the particle in the fluid among other particles in the fluid. The cross section of the at least one passage having greater than or equal to an average diameter of the particle may be useful for the particles that do not deform, compress or bend.

The cross-section of the at least one passage may be less than an average diameter of the particle in the fluid among other particles in the fluid. The cross section of the at least one passage having less than an average diameter of the particle may be useful for the particles that deform, compress or bend such as gel beads.

The particles in the fluid may include contaminants. The contaminants may be introduced by environment, such as airborne particulate matter, by consumables, such as pipette tips or by reagents, such as chemical compounds. The contaminants may be introduced by the environment, the consumables and the reagents during a step or a plurality of steps in processing of the sample.

The particles may be beads. In some cases, the beads may be porous, non-porous, solid, semi-solid, semi-fluidic, fluidic, and/or a combination thereof. In some instances, the beads may be dissolvable, disruptable, and/or degradable. In some cases, beads may not be degradable. In some cases, the bead may be gel beads. The gel beads may be hydrogel beads.

In some instances, the bead may comprise molecular precursors (e.g., monomers or polymers), which may form a polymer network via polymerization of the precursors. In some cases, a precursor may be an already polymerized species capable of undergoing further polymerization via, for example, a chemical cross-linkage. In some cases, a precursor can comprise one or more of an acrylamide or a methacrylamide monomer, oligomer, or polymer. In some cases, the bead may comprise prepolymers, which are oligomers capable of further polymerization. For example, polyurethane beads may be prepared using prepolymers. In some cases, the bead may contain individual polymers that may be further polymerized together. In some cases, beads may be generated via polymerization of different precursors, such that they comprise mixed polymers, co-polymers, and/or block co-polymers.

In some cases, a chemical cross-linker may be a precursor used to cross-link monomers during polymerization of the monomers and/or may be used to attach oligonucleotides (e.g., barcoded oligonucleotides) to the bead. In some cases, polymers may be further polymerized with a cross-linker species or other type of monomer to generate a further polymeric network or a matrix. Non-limiting examples of chemical cross-linkers (also referred to as a “crosslinker” or a “crosslinker agent” herein) include cystamine, gluteraldehyde, dimethyl suberimidate, N-Hydroxysuccinimide crosslinker BS3, formaldehyde, carbodiimide (EDC), SMCC, Sulfo-SMCC, vinylsilane, N,N′diallyltartardiamide (DATD), N,N′-Bis(acryloyl)cystamine (BAC), or homologs thereof. In some cases, the matrix may include polymer meshes, entangled polymers or similar structures to provide surface for attachment of reagents. In some aspects, hydrogen polymers may be useful as the matrix.

A bead may comprise natural and/or synthetic materials. For example, a polymer can be a natural polymer or a synthetic polymer. In some cases, a bead can comprise both natural and synthetic polymers. Examples of natural polymers include proteins and sugars such as deoxyribonucleic acid, rubber, cellulose, starch (e.g., amylose, amylopectin), proteins, enzymes, polysaccharides, silks, polyhydroxyalkanoates, chitosan, dextran, collagen, carrageenan, ispaghula, acacia, agar, gelatin, shellac, sterculia gum, xanthan gum, Corn sugar gum, guar gum, gum karaya, agarose, alginic acid, alginate, or natural polymers thereof. Examples of synthetic polymers include acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and/or combinations (e.g., co-polymers) thereof. Beads may also be formed from materials other than polymers, including lipids, micelles, ceramics, glass-ceramics, material composites, metals, other inorganic materials, and others.

The gel beads may be uniform or heterogeneous in cross-sectional dimensions. In some cases, the cross-sectional dimensions may have range between 1 micrometer (μm) to 1 millimeter (mm), 10 μm to 100 μm, 20 μm to 100 μm, 30 μm to 80 μm or 40 μm to 60 μm. Some examples of cross-sectional dimensions may include 30 μm, 40 μm, 50 μm, 55 μm, 57 μm, 60 μm, 64 μm, 70 μm, 72 μm, 75 μm, 100 μm, 125 μm, 150 μm, 200 μm, 250 μm, 500 μm, 750 μm and 1 mm.

The particles may be cellular components. The cellular components may include membranes, organelles, macromolecular components e.g., RNA, DNA, proteins, etc.). The cellular component may be released by lysis of cells or may be associated with the cells.

The particles may further be coupled to barcodes. The barcodes can be nucleic acid molecules with nucleic acid sequences. The barcodes across at least a subset of the particles are different. The barcodes can be assigned or associated with the at least a subset of particles to tag or label with unique identifiers. The nucleic acid sequences can include from 6 to about 20 or more nucleotides within a sequence of oligonucleotides.

The particles may be cells. The cells may be enclosed inside of or comprise a gel or a matrix (e.g., polymer matrix) to form a “cell bead.” The cell bead can contain cells or macromolecular constituents (e.g., RNA, DNA, proteins, etc.) of the cells. The cell bead may include a single cell or multiple cells, or a derivative of the single cell or multiple cells. For example after lysing and washing the cells, inhibitory components from cell lysates can be washed away and the macromolecular constituents can be bound as cell beads. In some cases, the cell bead may further comprise one or more field-attractable particles (e.g., paramagnetic particles, conductive particles, etc.), for facilitating sorting of the particles and further assaying. The field-attractable particles may be trapped in the gel matrix. In some instances, the field-attractable particles may be trapped evenly throughout the gel matrix. In some instances, the field-attractable particles may be trapped throughout the gel matrix such as to subject the whole of the cell bead evenly to a force (e.g., magnetic, electric) field.

The particles may be comprised in a population of particles. The population of particles may be homogeneous with similar or identical cross-sectional and/or vertical dimensions. The population of particles may be heterogeneous with dissimilar cross-sectional and/or vertical dimensions. Heterogeneity in the population of particles can be referred to as dispersity. The dispersity may influence flow characteristics of the fluid flowing through the at least one channel of the microfluidic device since the particles can have different flow characteristics due to dissimilar cross-sectional and/or vertical dimensions.

The dispersity may also influence blockage of the at least one channel. In some cases, the population of particles may be relatively monodisperse distribution. In particular, the dispersity of the particles described herein may have size distributions that have a coefficient of variation in their cross-sectional and/or vertical dimensions of less than 50%, less than 40%, less than 30%, less than 20%, and in some cases less than 15%, less than 10%, less than 5%, or less.

The dispersity may be determined by any suitable method. For example, high throughput measurements, e.g., measurements of 1000 or more particles, automated microscopy (e.g., using a Morphologi G3 system), dynamic imaging analysis (e.g., using a flow monitoring camera system), and light scattering (e.g., using a Mastersizer 3000 system), may be used.

The particles with a given cross-sectional dimension and/or vertical dimension may be produced using controlled preparation techniques, post preparation sorting and sizing techniques. For example, the given cross-sectional dimension and/or vertical dimension may be selected using size exclusion based separation techniques such as filtration or sieving techniques.

In some cases, nature of the particles may prevent use of size exclusion based separation techniques. For example, filtration techniques may be ineffective for the particles that are highly elastic or deformable since the particles may deform and pass through smaller openings than the given cross-sectional dimension and/or vertical dimension, resulting in dispersity. The particles that are deformable may also be more susceptible to blocking the channel.

In some cases, the population of particles may be subjected to alternate methods to separate the particles with a given cross-sectional dimension and/or vertical dimension. For example, the population of particles may include a first subset of particles with a first given cross-sectional dimension and/or vertical dimension along with a second subset of particles with a second given cross-sectional dimension and/or vertical dimension. Further, the first given cross-sectional dimension and/or vertical dimension may be smaller than the second given cross-sectional dimension and/or vertical dimension. The population of particles may be subjected to a flotation filtration technique where the fluid with the population of particles can be provided in a floatation chamber with an upward flow rate applied through the flotation chamber. The upward flow rate may be less than a gravitational acceleration vector of the second subset of the particles that may result in settling of the second subset of the particles while the first subset of the particles may float in the flotation chamber. The first subset of the particles can then be separated in the output reservoir. The particles may be separated on the basis of size and/or density.

In some cases, the particles with a given cross-sectional dimension and/or vertical dimension may be produced using size exclusion based separation techniques. For example, vector chromatography methods and systems can be used. In the vector chromatography systems, the channels or conduits with longer vertical dimensions can be provided for carrying the fluid with the particles. For example, the population of particles may include a first subset of particles with a first given cross-sectional dimension and/or vertical dimension along with a second subset of particles with a second given cross-sectional dimension and/or vertical dimension. Further, the first given cross-sectional dimension and/or vertical dimension may be smaller than the second given cross-sectional dimension and/or vertical dimension. The first subset of particles may traverse through the fluid faster compared to the second subset of particles and the first subset of particles may reach the output reservoir before the second subset of particles.

The particles may be of any suitable shape. Examples of particle shapes include, but are not limited to, spherical, non-spherical, oval, oblong, amorphous, circular, cylindrical, and variations thereof.

In an aspect, the present disclosure provide a system for filtering particles in a fluid may include a microfluidic device. The microfluidic device may further comprise a reservoir for holding the fluid comprising the particles, a channel in fluid communication with the reservoir and at least one filter feature in the channel. The at least one filter feature may comprise a field of a plurality of physical obstacles configured and arranged in the channel. The at least one filter feature may separate the particles in the fluid upon flow of the fluid from the reservoir through the channel.

For example, the at least one filter feature may retain the particles and permit the fluid to flow through, thereby separating the particles from the fluid. As another example, the at least one filter feature may separate the particles from contaminants (e.g., debris), to permit the fluid with particles to flow through with a decrease in an amount or concentration of the contaminants.

The channel may comprise a first end and a second end, wherein the first end is adjacent to the reservoir. A cross-section of the channel may decrease along a direction leading from the first end to the second end. The cross-section may be tapered. For example, the cross-section gradually decreases.

In addition to features of the microfluidic mentioned herein, the system may comprise a controller. The controller may be operatively coupled to the microfluidic device, wherein the controller subjects the fluid to flow from the reservoir to the channel. For example, the controller can be programmed to regulate flow options, e.g., the controller may instruct the microfluidic device to flow the fluid from the reservoir at a flow pressure and a flow rate. The controller can also be programmed to instruct the microfluidic device to release the fluid to the output reservoir. The controller can be programmed to direct the fluid from a reservoir to a filter feature. The controller can be programmed to regulate flow of the fluids in a first channel and a second channel. The fluids in the first channel and the second channel may be in fluid communication at an intersection.

### Methods

In an aspect, the present disclosure provides methods for filtering particles in a fluid. A method for filtering a particle in a fluid may comprise activating a microfluidic device comprising (i) a reservoir for holding the fluid comprising the particles, (ii) a channel in fluid communication with the reservoir, and (iii) at least one filter feature in the channel. The at least one filter feature may comprise a field of a plurality of physical obstacles configured and arranged in the channel, wherein the channel comprises a first end and a second end. The first end is adjacent to the reservoir. A cross-section of the channel decreases along a direction leading from the first end to the second end. The fluid may be subjected to flow from the reservoir to the channel. Upon flow of the fluid from the reservoir through the channel, the at least one filter feature separates the particles in the fluid. The fluid may be subjected to flow with the aid of a pressure increase or decrease along a direction leading from the first end to the second end.

In some embodiments, the method may include collecting the particles in an output reservoir. In some embodiments, method may further include directing the particles collected in the output reservoir to further processing. In some embodiments, the method may include directing the particles collected in the output reservoir to further processing.

In some embodiments, the method may include directing the particles to partitions. The partitions may be droplets. The partitions may be wells.

In some embodiments, the particles may include contaminants. In some embodiments, the particles may include beads. In some embodiments, the particles may include cellular components. In some embodiments, the particles may include cells. In some embodiments, the particles may include barcodes coupled thereto. The barcodes may be nucleic acid molecules comprising nucleic acid sequences. The barcodes across at least a subset of the plurality of particles may be different.

### Computer Control Systems

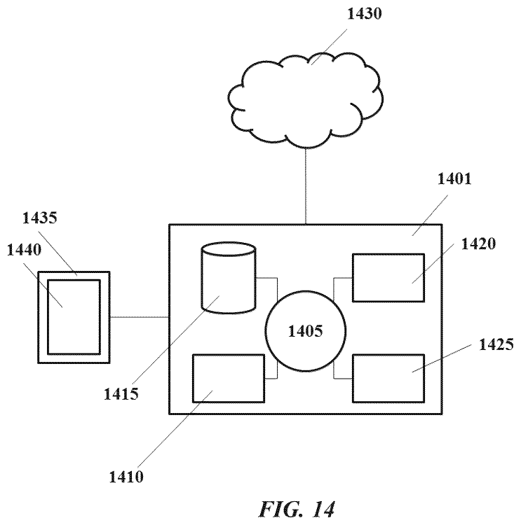

The present disclosure provides computer control systems that are programmed to implement methods of the disclosure. FIG. 14 shows a computer system 1401 that is programmed or otherwise configured to implement method of the disclosure including operation of a microfluidic system, activating a microfluidic device and subjecting a fluid to flow from a reservoir to a channel. The computer system 1401 can regulate various aspects of the apparatuses, systems and methods of the present disclosure, such as, for example, a flow rate of the fluid from the reservoir to a filter feature in a channel, a flow rate of the fluid from the channel to an output reservoir after filtration through eth filter feature, a fluid pressure of the fluid entering the filter feature, a fluid pressure of the fluid exiting the filter feature, a concentration of particles released in the output reservoir. The computer system 1401 can be an electronic device of a user or a computer system that is remotely located with respect to the electronic device. The electronic device can be a mobile electronic device.

The computer system 1401 includes a central processing unit (CPU, also “processor” and “computer processor” herein) 1405, which can be a single core or multi core processor, or a plurality of processors for parallel processing. The computer system 1401 also includes memory or memory location 1410 (e.g., random-access memory, read-only memory, flash memory), electronic storage unit 1415 (e.g., hard disk), communication interface 1420 (e.g., network adapter) for communicating with one or more other systems, and peripheral devices 1425, such as cache, other memory, data storage and/or electronic display adapters. The memory 1410, storage unit 1415, interface 1420 and peripheral devices 1425 are in communication with the CPU 1405 through a communication bus (solid lines), such as a motherboard. The storage unit 1415 can be a data storage unit (or data repository) for storing data. The computer system 1401 can be operatively coupled to a computer network (“network”) 1430 with the aid of the communication interface 1420. The network 1430 can be the Internet, an internet and/or extranet, or an intranet and/or extranet that is in communication with the Internet. The network 1430 in some cases is a telecommunication and/or data network. The network 1430 can include one or more computer servers, which can enable distributed computing, such as cloud computing. The network 1430, in some cases with the aid of the computer system 1401, can implement a peer-to-peer network, which may enable devices coupled to the computer system 1401 to behave as a client or a server.

The CPU 1405 can execute a sequence of machine-readable instructions, which can be embodied in a program or software. The instructions may be stored in a memory location, such as the memory 1410. The instructions can be directed to the CPU 1405, which can subsequently program or otherwise configure the CPU 1405 to implement methods of the present disclosure. Examples of operations performed by the CPU 1405 can include fetch, decode, execute, and writeback.

The CPU 1405 can be part of a circuit, such as an integrated circuit. One or more other components of the system 1401 can be included in the circuit. In some cases, the circuit is an application specific integrated circuit (ASIC).

The storage unit 1415 can store files, such as drivers, libraries and saved programs. The storage unit 1415 can store user data, e.g., user preferences and user programs. The computer system 1401 in some cases can include one or more additional data storage units that are external to the computer system 1401, such as located on a remote server that is in communication with the computer system 1401 through an intranet or the Internet.

The computer system 1401 can communicate with one or more remote computer systems through the network 1430. For instance, the computer system 1401 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 1401 via the network 1430.

Methods as described herein can be implemented by way of machine (e.g., computer processor) executable code stored on an electronic storage location of the computer system 1401, such as, for example, on the memory 1410 or electronic storage unit 1415. The machine executable or machine readable code can be provided in the form of software. During use, the code can be executed by the processor 1405. In some cases, the code can be retrieved from the storage unit 1415 and stored on the memory 1410 for ready access by the processor 1405. In some situations, the electronic storage unit 1415 can be precluded, and machine-executable instructions are stored on memory 1410.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1401, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1401 can include or be in communication with an electronic display 1435 that comprises a user interface (UI) 1440 for providing, for example, fluid control options (e.g., flow rate of the fluid, fluid pressure, concentration of particles, flow rate in the filter feature, flow rate in the output reservoir, etc.). Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1405. The algorithm can, for example, (i) filter a fluid with particles to sort into individual particles by separating aggregates using at least one filter feature, (ii) selectively filter a first subset of particles (e.g., debris) from a second subset of particles (gel beads) in a fluid, and/or (iii) sort individual gel beads into a plurality of droplets or an array of wells for further analyzing.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-85. (canceled)

86. A method for filtering particles in a fluid, comprising:
(a) providing a microfluidic device comprising:
(i) a reservoir configured to hold the fluid comprising the particles,
(ii) a channel in fluid communication with the reservoir, and
(iii) at least one filter feature disposed within the channel, the at least one filter feature comprising a plurality of physical obstacles arranged in the channel and at least one passage between the plurality of physical obstacles and through the filter feature, the passage having a cross-section of at least 30 μm;
wherein the channel comprises a first end and a second end, the first end being directly connected to the reservoir and disposed between the reservoir and the second end; and
wherein the at least one filter feature is (1) at least partially located in a tapered portion of the channel at the first end that decreases in cross-section along a direction from the first end to the second end, or (2) located in a portion of the channel comprising a contraction followed by an expansion, the contraction and expansion being between an upstream end and a downstream end of the plurality of physical obstacles of the at least one filter feature; and
(b) transporting the fluid from the reservoir through the at least one filter feature, wherein the particles are trapped by the at least one filter feature.

87. The method of claim 86, wherein the particles comprise a contaminant.

88. The method of claim 86, wherein the fluid further comprises a second type of particle that passes through the at least one filter feature in step (b).

89. The method of claim 88, wherein the second type of particle comprises beads, cells, or cellular components.

90. The method of claim 89, wherein the second type of particle comprises, or are enclosed in, a gel or a polymer matrix.

91. The method of claim 89, wherein the second type of particle comprises cellular components, and wherein the cellular components include one or more members selected from the group consisting of cell membrane, cell wall, organelles, macromolecular constituent, deoxyribonucleic acid (DNA), and ribonucleic acid (RNA).

92. The method of claim 88, wherein the second type of particle comprises particles having barcodes coupled thereto.

93. The method of claim 92, wherein the barcodes comprise nucleic acid molecules, each comprising a nucleic acid sequence.

94. The method of claim 92, wherein the barcodes differ across at least a subset of the second type of particle.

95. The method of claim 88, further comprising, after step (b), directing the second type of particle to one or more partitions.

96. The method of claim 95, wherein the one or more partitions comprises one or more droplets.

97. The method of claim 86, wherein the at least one filter feature is at least partially located in a tapered portion of the channel at the first end that decreases in cross-section along a direction from the first end to the second end.

98. The method of claim 86, wherein the at least one filter feature is located in a portion of the channel comprising a contraction followed by an expansion, the contraction and expansion being between an upstream end and a downstream end of the plurality of physical obstacles of the at least one filter feature.

99. The method of claim 86, wherein the microfluidic device further comprises a second channel that intersects the channel and a second reservoir in fluid communication with the second channel, wherein the method further comprises transporting a second fluid from the second reservoir through the second channel to the intersection.

100. The method of claim 99, wherein the second channel further comprises at least one filter feature that traps particles in the second fluid while the second fluid is transported from the second reservoir through the second channel to the intersection.

101. The method of claim 86, wherein the fluid is driven through the channel by a pressure increase or decrease along a direction leading from the first end to the second end.

102. The method of claim 86, wherein the fluid is an oil.

103. The method of claim 86, wherein the fluid is aqueous.

104. The method of claim 86, wherein the passage has a cross-section that is greater than or equal to a diameter of the particles.

105. The method of claim 86, wherein the passage has a cross-section that is less than a diameter of the particles.
